(19) United States
(12) Patent Application Publication
(10) Pub. No.: US 2005/0102106 A1
(43) Pub. Date: May 12, 2005

(54) Diagnostic method for assessing a condition of a performance animal

(75) Inventor: Richard Bruce Brandon, Brookfield (AU)

Correspondence Address: Colin G. Sandercock;Heller Ehrman White & McAuliffe LLP, Suite 300, 1666 K Street NW, Washington, DC, 20006, US

(73) Assignee: Genomics Research Partners Pty Ltd.

(21) Appl. No.: 10/630,988

(22) Filed: Jul. 28, 2003

Related U.S. Application Data
(63) Continuation of application No. 09/896,941, filed on Jun. 29, 2001, now abandoned.

(30) Foreign Application Priority Data
May 4, 2001 (AU) PR4809

(51) Int. Cl. G06F 19/00; G01N 33/48; G01N 33/50

(52) U.S. Cl. 702/20

(57) ABSTRACT

The condition and ability of an animal to perform to its best ability may be determined by correlating gene expression with clinical and other data. The methods include collecting biological samples and clinical history, generating digital results on gene expression levels in the samples, remotely accessing and comparing the results with information via a communications network. The invention provides methods for assessing a performance animal's condition by determining relative abundance of a target nucleic acid, accessing a remote database, correlating digital signals with information in the database, and reporting the condition of the animal. A diagnostic system comprising a microarray, a microarray reader, a database for storing information from the reader, and a server receiving digital signals from the reader is also disclosed. The reader determines the abundance of target nucleic acid, normalised to a reference nucleic acid, and generates a digital signal that may be displayed as a report.

### FIELD OF THE INVENTION

The present invention relates to a method for appraisal, assessment and/or diagnosis of a condition of a performance animal and its capacity to perform to its best ability. The invention particularly relates to a method applicable when current blood tests are not capable of detecting or classifying a condition.

### BACKGROUND OF THE INVENTION

A condition of a performance animal, for example a racehorse, can be currently determined by conventional means such as a blood profile test and clinical appraisal. However, these tests are of limited value because a correlation between results of a blood profile test or clinical appraisal and a condition or state of a performance animal is minimal.

A blood profile test may be suitable for providing some information in relation to an animal that is clinically diseased or ill, but is rarely suitable for determining a level of performance of an animal, particularly if the animal is healthy according to use of current clinical appraisal methods. Although blood profile tests are relatively inexpensive and easy to perform, they do not provide assessment of a wide range of conditions, correlations between test results and conditions of animals are poor, are limited to assessment of a few diseases, and are sometimes only useful in assessment of advanced stages of disease where clinical intervention is too late to prevent significant loss of performance.

Alternative diagnosis or assessment procedures are often complex, invasive, inconvenient, expensive, time consuming, may expose an animal to risk of injury from the procedure, and often require transport of the animal to a diagnostic centre. In many instances there is no overt disease, or the animal is healthy, and the procedure is simply performed to gain further information about the capacity of a performance animal to perform to its best ability. Diagnostic methods may be used to determine severity of a sub-clinical disease, its possible effect on performance, whether training should persist, level of risk associated with continued training and whether continued training may adversely affect future performance. Factors including subtle changes in diet, training regime, stable, or season may affect performance of an animal.

Diagnosing a disease or determining risk of a disease using genetic means is known but has limitations. For example, the cause of combined immunodeficiency disease (CID) in Arabian horses is known to be genetically based. A horse heterozygous for CID (containing a normal and abnormal copy of the gene for DNA-dependent protein kinase catalytic subunit) is described in U.S. Pat. No. 5,976,803. Such a horse will pass on the normal and abnormal copies of the gene to its offspring. Two heterozygous horses will produce a foal with a one in four chance of having two abnormal copies of the gene (clinical CID resulting in death). The abnormal copy of the gene can be detected in DNA isolated from the animal using a DNA-based diagnostic test such as polymerase chain reaction (PCR). Such a test uses specific DNA primers to amplify different size amplification products for the normal and abnormal versions of the gene. The amplification products can be easily distinguished by size separation on an agarose gel.

In this example, the gene responsible for CID and the exact DNA sequence of the normal and abnormal genes are known. However, in many instances conditions and disease are caused by unknown genes, or through contributions from many genes. Alternatively, genes may be suspected of being a cause of a condition but not yet proven, or the gene may be known but the exact nucleotide sequence or abnormality in the gene causing a condition is not known. Accordingly, genetic testing as described by the above example is of limited value.

Other genetic tests include determining relative levels of gene expression using microarrays. Such tests have been used to determine specific genes that are differentially expressed in normal and diseased tissue. This has been used to assess a condition of a patient and is described in U.S. Pat. No. 6,194,158 which relates to gene expression in relation to brain cancers such as glioblastoma. A nucleic acid identified in such a manner and described in this patent may encode a complete or partial gene of interest, which may be attached to a substrate, for example a microarray, to assess relative gene expression of the differentially expressed gene. A further extension of the use of relative gene expression technology has been used in diagnosis (class prediction), sub-classification (class discovery) and subsequent choice of therapy of leukemic cancer in human (Golub, 1999, Science 286 531), herein incorporated by reference. Diagnosis and sub-classification of disease is possible in these examples because a limited number of genes are differentially expressed, the condition is well defined, current tests can be used to diagnose and classify the disease and/or symptoms are clinically obvious. In contrast, determining a condition of a performance animal relies on detection of differential expression of a large number of genes and correlation to data collected from a large number of samples where the clinical condition of the animals has been well documented and is not necessarily either clinically obvious, or current tests show no definitive diagnosis or classification of disease.

U.S. Pat. No. 6,114,114 relates to a method for comparing relative abundance of gene transcripts between healthy and diseased human tissue by use of high-throughput sequence-specific analysis of individual RNAs or their corresponding cDNAs. This provides a method and system for quantifying relative abundance of gene transcripts in a biological sample. A diagnostic test can be performed on an ill patient in whom a diagnosis has not been made. The patient's sample is collected, gene transcripts isolated and expanded to an extent necessary for gene identification and determination of the relative abundance of individual gene transcripts. Optionally, the gene transcripts are converted to cDNA and then the relative abundance determined. A sample of the gene transcripts are subjected to sequence-specific analysis and quantified. These gene transcript sequences are compared against a reference database of the relative abundance of specific genes and their DNA sequences in diseased and healthy patients. The patient may be diagnosed as having a disease(s) with which the patient's data set most closely correlates. Because diseases are mostly species specific, due to variations in gene sequence between species, and due to variations between species in the relative abundance of different RNAs in tissues, the method described in U.S. Pat. No. 6,114,114 is limited to available databases comprising information in relation to gene expression in disease in human. This patent describes identification of individual genes that are differentially expressed in abnormal and normal tissues. The patent does not describe the detection or diagnosis of a condition in performance animals based on a pattern of gene expression or differences in gene expression.

A method for a medical diagnostic advice system accessible via a computer network is described in U.S. Pat. No. 6,206,829. This method provides medical diagnosis of a condition based in part on a patient's history and patient provided description of symptoms. This method is not useful for conditions which require detailed physical examination and/or laboratory testing to provide a diagnosis. For example, this method is not suitable for diagnosing a condition which is not readily or physically detectable. In particular, this method would not be useful in diagnosing a condition in an otherwise healthy appearing individual, in a normal individual according to clinical appraisal and current diagnostic methods, or in an individual requiring differentiating information in relation to its level of performance, or in animals not capable of communicating information on a clinical history. This method also does not describe use of molecular biological methods, for example assessment of gene expression, in diagnosis.

The prior art describes methods for diagnosing disease using standard blood tests, which are limited to testing a few diseases and may have low sensitivity and specificity, and low correlation to a condition. Invasive procedures are available for more accurate assessment for a broader range of diseases, however, such methods have inherent risks, are costly and time consuming. Genetic methods for diagnosing disease are often limited to specific genes that have been identified which correlate with particular diseases. Genetic diagnostic methods may also be limited to human application because of dependence of such methods on information provided by the patient, information available in relation to a specific disease, species and/or specific DNA sequence information.

The abovementioned prior art does not describe a method for testing for a condition, level of performance, response to or detection of drugs, sub-classifying known disease, identification of new pathological descriptions of diseases or stages of diseases in a performance animal. In particular, the prior art does not provide a rapid method for diagnosing a condition using data remotely stored and accessible via a communications network, for example an intranet, the Internet or extranet, including wireless transmission.

### SUMMARY OF THE INVENTION

It is an object of the present invention to provide a relatively inexpensive, accurate, clinically correlative, convenient, rapid and preferably minimally invasive method for providing assessment information for a condition, and ability of an animal to perform to its best ability.

The invention relates to a method for measuring levels of gene expression, preferably in cells found in blood, and correlating gene expression with clinical and other relevant data to assess/appraise/diagnose a condition of a performance animal. The method includes the steps of collecting a biological sample and clinical history, testing the sample to produce digital results on the relative levels of gene expression, remotely accessing and comparing the results with information via a communications network, and providing a report in relation to the condition or state of the performance animal.

In one aspect the invention provides a method for assessing a condition of a performance animal including the steps of:
(a) determining in a sample from a performance animal a relative abundance of a target nucleic acid normalised to a reference nucleic acid and providing the relative abundance of the target nucleic acid as a digital signal;
(b) accessing a remotely located database comprising digital information in relation to relative abundance of the target nucleic acid which corresponds to a particular condition of the performance animal;
(c) correlating the digital signal of step (a) with the digital information of step (b) thereby identifying a particular condition of the performance animal; and
(d) reporting the particular condition of the performance animal.

The database is preferably accessible via a communications network.

More preferably, the communications network comprises the Internet, an intranet, an extranet or wireless means.

In one embodiment of the method, the step of determining the relative abundance of the target nucleic acid includes the steps of:
(i) detecting a hybridised complex formed by at least one target nucleic acid and a complementary nucleic acid located on a solid support to provide a digital target sample signal;
(ii) detecting a hybridised complex formed by at least one reference nucleic acid and a complementary nucleic acid located on a solid support to provide a digital reference sample signal; and
(iii) comparing the digital target sample signal of step (i) and the digital reference sample signal of step (ii) to provide a digital signal of relative abundance of the target sample.

The complementary nucleic acids of step (i) and step (ii) may comprise a same or homologous nucleotide sequence.

Preferably, the hybridised complex of step (i) and step (ii) is detected by respectively labelling the target and the reference nucleic acid.

More preferably, the respective labelled nucleic acid is labelled with Cy3 or Cy5.

Preferably, the respective labelled nucleic acid is cDNA.

The respective target nucleic acid and reference nucleic acids may be concurrently hybridised with respective complementary nucleic acids.

The solid support is preferably an array.

More preferably, the array is a microarray.

The performance animal is preferably a mammal.

More preferably, the mammal is human, horse, dog or camel.

The performance of an animal may relate to its athletic ability and any condition that may enhance, hinder, impede or not change its expected ability.

The condition may enhance, hinder, impede or not change an expected ability of the performance animal.

The condition of the performance animal may comprise normal, pre-clinical disease, overt disease, progress and/or stage of disease, undiagnosed or unclassified conditions, presence of drugs, response to drugs, response to exercise, response to vaccines, therapies, nutritional states and response to environmental conditions.

The disease may comprise laminitis, lameness, viral disease, colic, gastritis, gastric ulcers, respiratory ailments and epistaxis.

Another aspect of the invention relates to a diagnostic system comprising:
(A) a microarray comprising respective nucleic acids complimentary to a target nucleic acid and reference nucleic acid;
(B) a microarray reader that detects hybridised complexes formed respectively by the target nucleic acid and the reference nucleic acid with their complimentary nucleic acids and generates a digital signal;
(C) a database storing information in relation to relative abundance of the target nucleic acid corresponding to a particular condition of a performance animal;
(D) a diagnostic server that receives the digital signal and correlates the digital signal with information in the database to identify said particular condition and reports said particular condition; and
(E) a means for communicating between the microarray reader and the diagnostic server.

The microarray reader may determine relative abundance of the target nucleic acid normalised to the reference nucleic acid and generate a digital signal for the relative abundance of the target nucleic acid.

The means of communication may be a network.

The diagnostic system may further comprise a means to display the report.

The present invention has advantages over current methods for diagnosing disease, for example laminitis (inflammation of the soft tissues in the hoof) in a racehorse. In many instances laminitis is sub-clinical, that is, the horse does not present clinically as lame. However, an owner or trainer may be concerned that the horse is not performing to the best of its ability. In this instance, a blood test and/or X-ray may traditionally be performed. However, subtle inflammation of the hoof will not be able to be detected by X-ray and will not be reflected in any abnormal values in current blood tests. Considerable expense through current test costs and lost training time, and inconvenience through transport of animals to diagnostic centres could be encountered with the risk of gaining little information on the exact condition or state of the animal, and whether and when it can perform to the best of its ability. Hence, the horse may have normal results from current tests, but actually have laminitis and thereby may not be performing to its best ability, and the owner and trainer would remain oblivious to its condition.

Another example of deficiencies of current blood tests is evident by methods for testing an athlete for use of illegal or prohibited performance-enhancing steroids. Current blood tests directly measure a level of a steroid in serum using equipment such as high performance liquid chromatographs, gas chromatographs or similarly sensitive equipment. These tests are not capable of detecting the steroid where the athlete is also using masking drugs, or where the athlete has not taken steroids for a period prior to the test being performed.

It will be appreciated that the present invention may have advantages of being relatively inexpensive, accurate, convenient, rapid and minimally invasive. Further, the present invention is not dependent on isolating a known gene to determine a condition of an animal. The present invention may be used with a nucleic acid of known nucleotide sequence and expression level (gene transcript relative abundance) in a reference sample which is comparable with a nucleic acid expression level in a test sample.

### BRIEF DESCRIPTION OF THE FIGURES

FIG. 1 is a flow diagram showing steps for diagnosing a condition of an animal in accordance with the invention;

FIG. 2 is a diagram illustrating an environment for working the invention as shown in FIG. 1;

FIG. 3 is a flow diagram illustrating steps for preparing an array in accordance with an embodiment of the invention;

FIG. 4 is a flow diagram showing steps for determining a nucleic acid expression level in a biological sample; and

FIG. 5 is a flow diagram illustrating steps for building a database in accordance with an embodiment of the invention.

### DESCRIPTION OF PREFERRED EMBODIMENTS

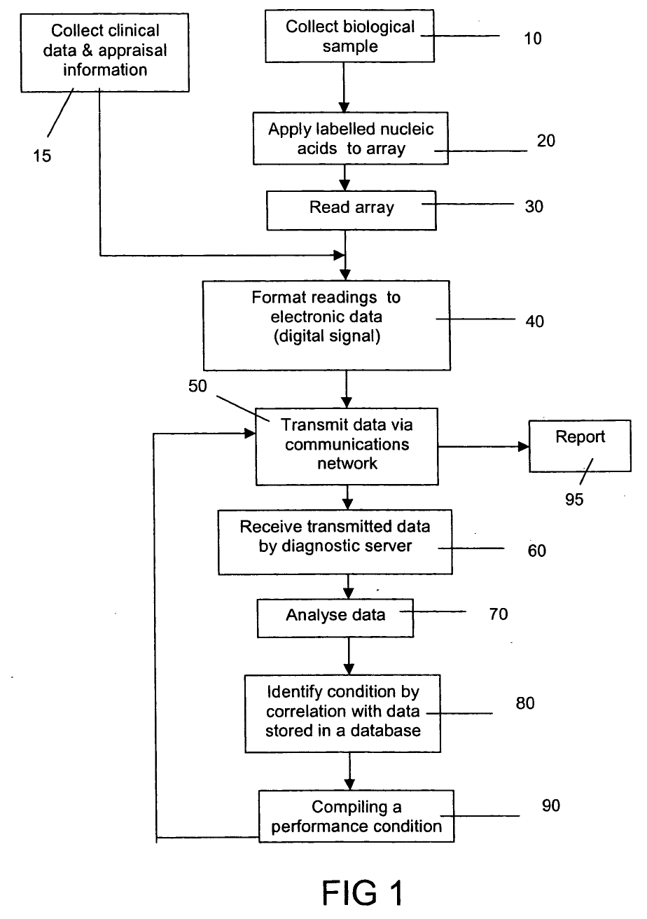

FIG. 1 is a flow diagram of one embodiment of the invention showing steps for assessing a biological sample for diagnosing or assessing a condition of an animal. A user collects a biological sample 10, for example a blood sample from a horse. At the same time, clinical data and appraisal information is collected in a standard format 15, for example by filling in a form. The biological sample 10 is processed so that nucleic acids contained therein are detectable when hybridised with a complementary nucleic acid located on an array 20. The nucleic acid may be detectable by a label incorporated therein. Preferably, the array 20 is a microarray which is read 30 by standard methods and equipment common to the art to identify and measure relative abundance of those nucleic acids from the biological sample which have bound to the microarray 20 (inclusion of a reference sample run in parallel allows for the calculation of the relative abundance of target nucleic acids). Data from the read microarray 30 and clinical data and appraisal information 15 is formatted 40 and transmitted via a communications network 50, for example the Internet, to a diagnostic server 60. The transmitted data is analysed 70, for example by comparison to a database of previously collected information in relation to expression levels (relative abundance) of the nucleic acids applied to the microarray 20. The analysis enables correlation to a condition 80. In this manner, the expression levels (relative abundance) of the nucleic acids applied to the microarray 20 are correlated with previously collected data relating to known conditions stored in a database 80 and compiled 90. The database may also store information in relation to an identity of known nucleic acids, nucleotide sequence on the array and/or location of nucleic acids on the array. Results in relation to health and performance condition are transmitted via a communications network 50 and may also be provided to the user as a report 95, for example a hardcopy printout or visually on a computer monitor. The steps are described in more detail hereinafter.

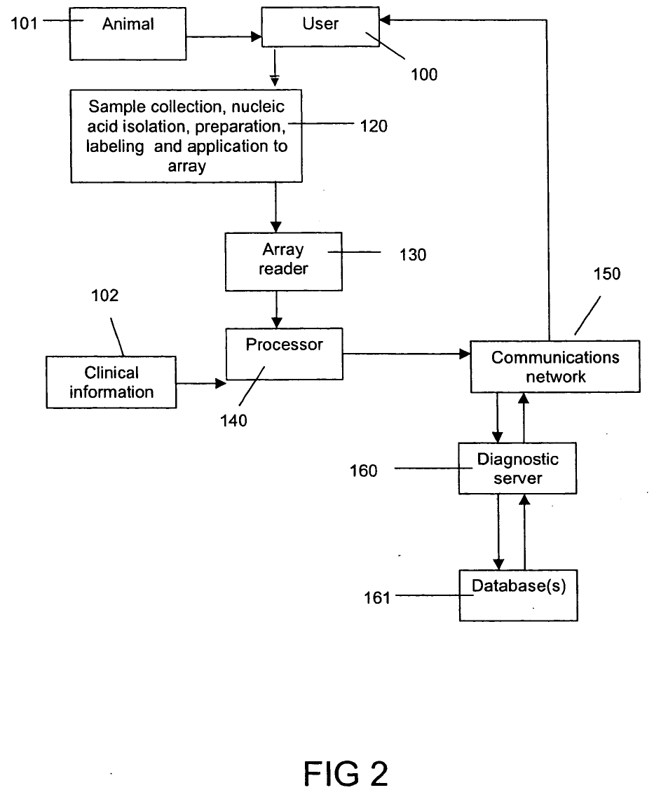

FIG. 2 shows an environment for working the method described in FIG. 1. A user 100, which may be a vet or practitioner, collects a sample 120 from an animal 101, for example a blood sample from a horse or athlete. Concurrently, information in relation to a condition of the animal is collected in a standard format 102. The sample is collected, nucleic acids isolated therefrom, prepared and applied to an array 120 and the array is read by an array reader 130. Data from the array reader 130 and clinical appraisal and condition information 102 is entered into a computer and formatted by a processor 140, which may be for example, a laptop computer with a modem. The formatted data is transmitted via a communications network 150, for example the Internet. A diagnostic server 160 receives the transmitted data and the data is compared with a database(s) 161 which stores data, for example, data in relation to nucleic acid location on an array, expression level (relative abundance) of a nucleic acid hybridised with a corresponding nucleic acid on an array, and data correlating nucleic acid expression level and performance, health, or condition of an animal.

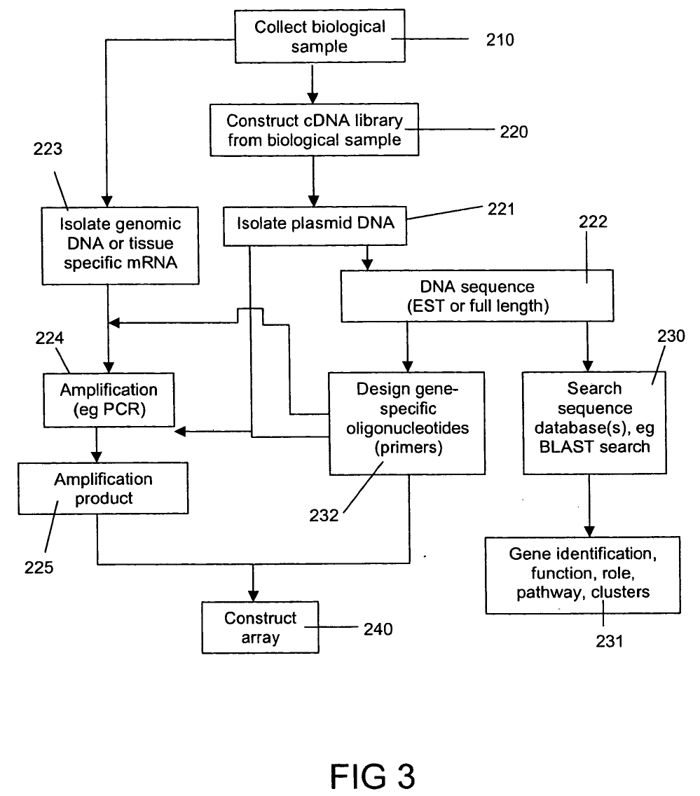

FIG. 3 is a flow diagram illustrating steps for preparing an array in accordance with the invention. A biological sample 210 is collected from an animal. Biological sample 210 may comprise for example, a blood sample (preferably white blood cells isolated therefrom), urine sample or tissue sample (including fetal tissues and tissues from stages of development). A specific aim of collecting the biological sample is to isolate and sequence as many relevant genes from the sample for use on an array. Nucleic acids are isolated from the biological sample. In one instance the sample is used to prepare genomic DNA or tissue specific mRNA 223. In another instance RNA is isolated from the biological sample 210 and a cDNA library 220 is prepared from the isolated RNA. Plasmids 221 comprising cDNA inserts from library 220 may be sequenced 222 from either or both 5′ and/or 3′ end of the nucleic acid. Preferably, sequencing is from the 3′ end. Sequences may comprise Expressed Sequence Tags (EST). If an isolated nucleic acid does not encode a full-length gene (eg. an EST), a partial nucleic acid may be used as a probe to isolate a full-length nucleic acid. Alternatively, or in addition, EST sequence information may be compared directly with a sequence database 230, for example GenBank, and a search for related or identical sequences performed. Putative gene identification and function 231 may be determined from a search, for example a BLAST search performed in step 230. By determining the number of times each gene is represented in the library, a computer may be programmed to enable the normalisation and standardisation of the relative abundance data of mRNAs in a sample.

Gene-specific oligonucleotides 232 may be synthesised using information from EST or full-nucleotide sequence 222 data. Gene-specific oligonucleotides 232 may be used as amplification primers to amplify (step 224) a region of a corresponding nucleic acid. The nucleic acid used as template to amplify a region of corresponding nucleic acid may be, for example, isolated plasmid DNA 221 and/or genomic DNA, cDNA or mRNA (eg. used with RT-PCR) 223. The nucleic acid thus prepared can be used directly as the nucleic acids for attaching to an array 240. Amplification products 225 may also be generated using non-gene-specific primers (eg. oligo-dT, plasmid sequence flanking a nucleic acid of interest). Oligonucleotides corresponding to a gene 232 may also be used on array 240.

In one embodiment, the step relating to constructing cDNA 220 and isolating plasmids 221 comprising the cDNA may be omitted. In this embodiment, isolated genomic DNA or tissue specific mRNA 223 is used as a template to make amplification product 225 by amplification using gene-specific primers 232. Amplification product 225 may be attached to array 240.

Nucleic acids attached to array 240 preferably represent most, more preferably all, expressed genes in a given tissue from an animal of interest.

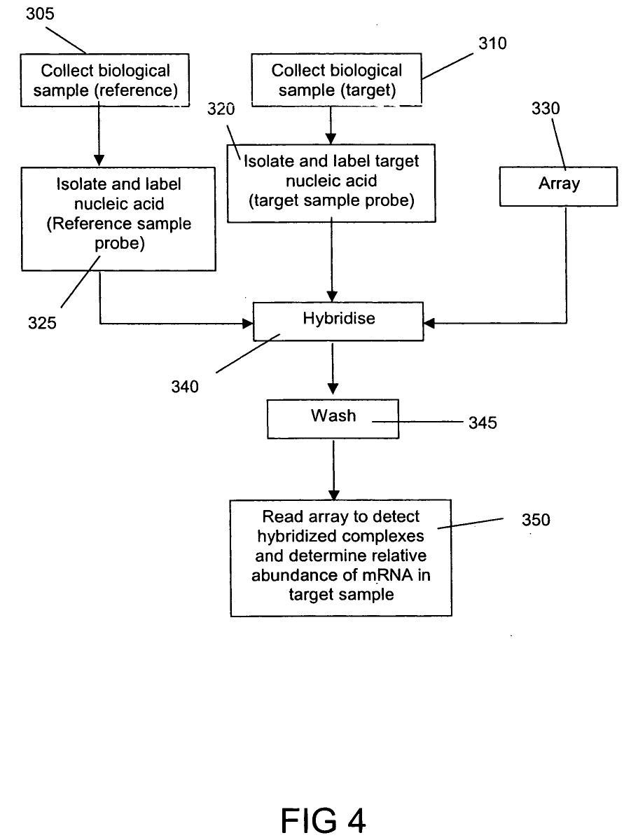

FIG. 4 shows a flow diagram comprising steps for determining gene expression in biological samples comprising both the reference 305 and target 310 samples. Nucleic acids, in particular RNA (total RNA or mRNA), are isolated from biological samples 305 and 310. cDNA is prepared from the RNA and the cDNA is labelled resulting in probes 320 and 325. Alternatively, or in addition, cDNA may be used as a template to synthesise labelled antisense RNA for use as probes 320 and 325. Reference sample probe 325 may be provided as a previously prepared probe of known concentration. Accordingly, reference sample probe 325 need not be synthesised in parallel with each target sample probe. Internal controls for reference sample probe 325 and target sample probe 320 provide a means for normalising and scaling relative probe concentrations.

Test sample probe 320 and reference sample probe 325 are hybridised with array 330 in step 340. Array 330 may, for example, have been prepared by steps shown in FIG. 3. The hybridised array is washed 345 to remove non-specific hybridisation of probes 320 and 325. It will be appreciated that one skilled in the art could select different stringency conditions of wash 345 as required. Array 330 is read in an array reader 350 to determine relative abundance of RNA in the original sample, which correlates with expression of the corresponding gene in the biological sample.

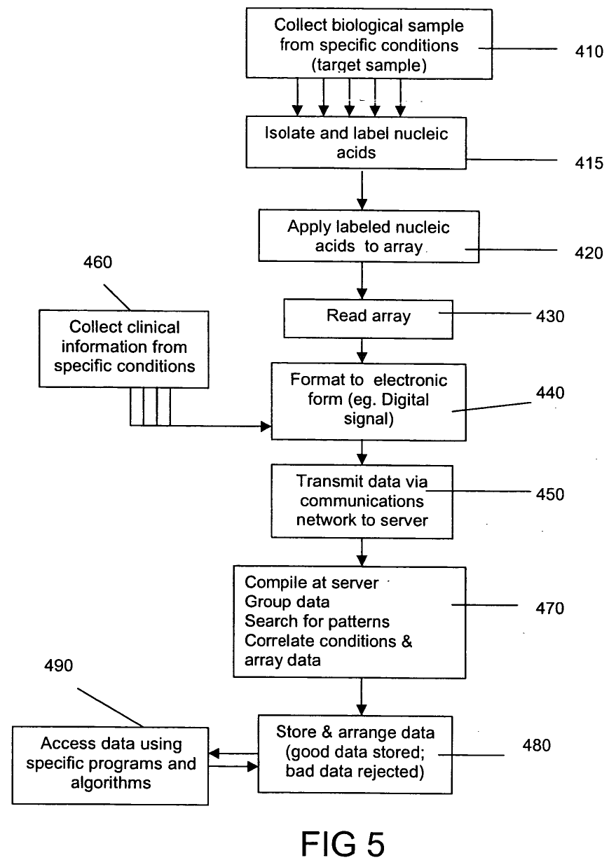

FIG. 5 is a flow diagram illustrating steps for building a database in accordance with the invention. Biological samples 410 are collected from animals having specific known condition(s). Preferably, about 1,000 biological samples 410 are collected from normal animals to establish a normal reference range of relative nucleic acid abundance levels. Nucleic acids are isolated and labelled 415 from sample 410. The labelled nucleic acids 415 are applied to array 420, which may be prepared as described in FIG. 3. The array is read 430 and data formatted 440 into an electronic form, for example a digital signal, suitable for transmission via a communications network 450. Clinical information from clinical appraisal, in relation to conditions of animals of interest is measured, documented and compiled 460. The clinical information is preferably collected in a standard format, for example, a white blood cell count over a specified level may be given a number (for example between 1-10), and specific histopathological conditions will be graded (for example between 1-10). Conditions may include disease, response to drugs, training, nutrition and environment. The clinical information 460 is formatted into electronic form 440, for example a digital signal, suitable for transmission via a communications network 450.

The process is repeated such that a collection of several array readouts for particular conditions are made. A standard range (for example, a population median of 95%) of values for each of the represented genes and its relative abundance can be calculated. This reference range can then be used as a comparison to test sample results.

Nucleic acid expression information from a read array 430 for a target sample is correlated with previously measured conditions 460 to provide information on nucleic acid expression level (relative abundance) with any previously measured condition. This information is compiled at server 470 and good data is stored and bad data rejected 480. The compilation process includes collection of a large enough set of array readout information for a particular condition so that statistical calculations can be made. The compilation 470 may also include use of sophisticated pattern recognition and organisational software and algorithms (examples common to the art include algorithms such as K means, Nova and Mann Whitney, Self Organising Maps, principal component analysis, hierarchical clustering—any one of which is available as part of proprietary software packages) such that expression patterns that differ to normal or expected condition can be identified. Concurrently, comprehensive clinical information 460 for animals may be collected and biological samples 410 tested on arrays so that correlations can be made between any clinical observation and array data. In this manner a database is created comprising data on nucleic acid expression which may include data correlating any desired condition, for example normal and specific abnormal condition(s), with nucleic acid expression. The stored data 480 may be accessed using specific programs and algorithms 490.

Definitions

Unless defined otherwise, all technical and scientific terms used herein have the meaning as commonly understood by those of ordinary skill in the art to which the invention belongs. Although any methods and materials similar or equivalent to those described herein can be used in the practice or testing of the present invention, preferred methods and materials are described. For the purpose of the present invention, the following terms are defined below.

The term “nucleic acid” as used herein designates single or double stranded total RNA, mRNA, RNA, cRNA and DNA, said DNA inclusive of cDNA and genomic DNA.

The term nucleic acid also comprises modifications, for example, chemical base substitutions and nucleic acid comprising a polyamide backbone such as peptide nucleic acids (PNAs) as described in International Patent WO 92/20702 and (Egholm, et al., 1993, Nature, 365, 560) herein incorporated by reference. It will also be appreciated that the backbone of a nucleic acid may comprise a peptide-like unit as well as a unit of sugar groups linked by phosphodiester bridges, optionally substituted with other groups such as phosphorothioates or methylphosphonates.

The term “isolated nucleic acid” as used herein refers to a nucleic acid subjected to in vitro manipulation into a form not normally found in nature. Isolated nucleic acid includes both native and recombinant (non-native) nucleic acids.

An “oligonucleotide” has less than eighty (80) contiguous nucleotides, whereas a “polynucleotide” is a nucleic acid having eighty (80) or more contiguous nucleotides. An oligonucleotide may be used for example as a probe, primer or attached to a substrate as an array element.

A “probe” may be a single or double-stranded oligonucleotide or polynucleotide, suitably labelled for the purpose of detecting a complementary nucleotide sequence of a nucleic acid which may be attached to a solid support, for example a microarray. Useful labels include, for example, Cy3 and Cy5. A single stranded probe may be synthesised from cDNA thereby making antisense RNA.

A “primer” is usually a single-stranded oligonucleotide, preferably having 20-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. The invention in one embodiment uses oligo-dT primers which may anneal to a polyA region of mRNA. In another embodiment, gene-specific primers may be used which anneal to complementary isolated nucleic acid from a biological sample, to amplify nucleotides therebetween. Use of these primers is provided in more detail hereinafter.

Nucleic Acid Sequence Comparison

Terms used herein to describe sequence relationships between respective nucleic acids include “comparison window”, “sequence identity”, “percentage of sequence identity” and “substantial identity”. Optimal alignment of sequences for aligning a comparison window may be conducted by computerised implementations of algorithms (for example ECLUSTALW and BESTFIT provided by WebAngis GCG, 2D Angis, GCG and GeneDoc programs, incorporated herein by reference) or by inspection and the best alignment (i.e., resulting in the highest percentage homology over the comparison window) generated by any of the various methods selected.

Reference may be made to the BLAST family of programs as for example disclosed by Altschul et al., 1997, Nucl. Acids Res. 25 3389, which is incorporated herein by reference. A detailed discussion of sequence analysis can also be found in Chapter 19.3 of CURRENT PROTOCOLS IN MOLECULAR BIOLOGY Eds. Ausubel et al., (John Wiley & Sons, Inc. 1995-1999).

The term “sequence identity” is used herein in its broadest sense to include the number of exact nucleotide matches having regard to an appropriate alignment using a standard algorithm, having regard to the extent that sequences are identical over a window of comparison. “Sequence identity” may be understood to mean the “match percentage” calculated by the DNASIS computer program (Version 2.5 for windows; available from Hitachi Software engineering Co., Ltd., South San Francisco, Calif., USA).

As generally used herein, a “homolog” shares a definable nucleotide sequence relationship with a nucleic acid.

In one embodiment, nucleic acid homologs share at least 60%, preferably at least 70%, more preferably at least 80%, and even more preferably at least 90% sequence identity with the nucleic acids of the invention.

In yet another embodiment, nucleic acid homologs hybridise to nucleic acids under at least low stringency conditions, preferably under at least medium stringency conditions and more preferably under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

In DNA, complementary bases are:
(i) A and T; and
(ii) C and G.

In RNA, complementary bases are:
(i) A and U; and
(ii) C and G.

In RNA-DNA hybrids, complementary bases are:
(i) A and U;
(ii) A and T; and
(iii) G and C.

Modified purines (for example, inosine, methylinosine and methyladenosine) and modified pyrimidines (thiouridine and methylcytosine) may also engage in base pairing. Hybridise and hybridisation may also refer to pairing between complimentary modified nucleic acids for example PNA and DNA, and PNA and RNA respectively.

A nucleic acid probe and complementary nucleic acid located on an array may hybridise with each other.

“Stringency” as used herein, refers to temperature and ionic strength conditions, and presence or absence of certain organic solvents and/or detergents during hybridisation. The higher the stringency, the higher will be the required level of complementarity between hybridising nucleotide sequences.

“Stringent conditions” designates those conditions under which only nucleic acid having a high frequency (percentage) of complementary bases will hybridise.

Stringent conditions are well known in the art, such as described in Chapters 2.9 and 2.10 of Ausubel et al., supra, which are herein incorporated by reference. A skilled addressee will also recognise that various factors can be manipulated to optimise the specificity of the hybridisation. Optimisation of the stringency of the final washes can serve to ensure a high degree of hybridisation.

As used herein, an “amplification product” refers to a nucleic acid product generated by nucleic acid amplification techniques.

Suitable nucleic acid amplification techniques are well known to the skilled addressee, and include PCR as for example described in Chapter 15 of Ausubel et al. supra, which is incorporated herein by reference; strand displacement amplification (SDA) as for example described in U.S. Pat. No. 5,422,252 which is incorporated herein by reference; rolling circle replication (RCR) as for example described in Liu et al., 1996, J. Am. Chem. Soc. 118 1587 and International application WO 92/01813; International Application WO 97/19193, which are incorporated herein by reference; nucleic acid sequence-based amplification (NASBA) as for example described by Sooknanan et al., 1994, Biotechniques 17 1077, which is incorporated herein by reference; ligase chain reaction (LCR) as for example described in International Application WO89/09385 which is incorporated herein by reference; and Q-β replicase amplification as for example described by Tyagi et al., 1996, Proc. Natl. Acad. Sci. USA 93 5395 which is incorporated herein by reference. Preferably, amplification is by PCR using primers and nucleic acids as described herein.

The term “array” refers to an ordered arrangement of hybridisable array elements. The array elements are arranged so that there are preferably multiple copies of a single element as an internal control, enough copies of the single element to specifically and sensitively hybridise to its complementary nucleic acid, and preferably at least one or more different array elements, more preferably at least 10 array elements, and even more preferably at least 100 array elements, and most preferably at least 5,000 array elements on a substrate surface. Where an array surface is small, for example 1 cm², the array may be referred to as a “microarray”. Furthermore, hybridisation signal from respective array elements is individually distinguishable. In one embodiment, an array element comprises a polynucleotide sequence. In another embodiment, an array element comprises an oligonucleotide sequence.

“Element” or “array element” in an array context, refers to a hybridisable nucleic acid arranged on a surface of a substrate.

“Biological sample” is used in its broadest sense and may comprise a tissue, for example from a biopsy; bodily fluid, for example blood, sputum, urine, bronchial or nasal lavages, joint fluid, peritoneal fluid, thoracic fluid; a cell; an extract from a cell, for example, an organelle or nucleic acid inclusive of a chromosome, genomic DNA, RNA (total and mRNA), and cDNA.

A “blood profile test” is defined herein as use of current technology to assess blood of an animal, and may include cell counts, cell appraisal and other biochemical, immunological and cellular tests.

“Clinical appraisal” is defined herein as use of observation, experience and/or use of more sophisticated diagnostic techniques. Alternative diagnostic techniques used to gain more information on conditions of performance animals include tests on lavages taken from body cavities, urine tests, bronchoscopy, ultrasound, MRI, CAT scans, X-rays, scintigraphy, and investigative surgery and tissue biopsy.

A “condition or state of an animal” refers to any influence, external or internal, that may hinder, enhance or not change the capacity of an animal to perform to its best ability.

The term “up-requlated” refers to mRNA levels encoding a gene which are detectably increased in a biological sample from a test animal compared with mRNA levels encoding the same gene in a biological sample from normal animal.

The term “down-regulated” refers to mRNA levels encoding a gene which are detectably decreased in a biological sample from a test animal compared with the mRNA levels encoding the same gene in a biological sample from normal animal.

The term “normal” is used herein to refer to an animal which does not have any visible abnormalities or known performance hindrance or enhancement, as detected by an assessment by for example, a trainer, owner(s), own person, veterinarian, practitioner, independent authorities or bodies or through the use of for example a clinical appraisal, routine blood profiles, current available diagnostic technologies.

Throughout this specification, unless the context requires otherwise, the words comprise, comprises and comprising will be understood to imply the inclusion of a stated integer or group of integers but not the exclusion of any other integer or group of integers.

In order that the invention may be readily understood and put into practical effect, particular preferred embodiments will now be described by way of the following non-limiting examples.

### Step 1

Biological Sample Collection

A biological sample comprising nucleic acids, for example total RNA and mRNA, is collected. The biological sample may include cells at various stages of development, differentiation and activity. The biological sample in most instances would be whole blood collected from a vein of a performance animal. However, the biological sample may include a fluid and/or tissue, for example sputum, urine, tissue biopsies, bronchial or nasal lavages, joint fluid, peritoneal fluid or thoracic fluid which comprises cells. Cells present in blood which comprise mRNA include neutrophils, lymphocytes, monocytes, reticulocytes, basophils, eosinophils, macrophages. All of these cell types also appear in tissues of non-blood origin at various times in various conditions. Methods described herein may include use of the abovementioned cell types. The biological sample is collected and prepared using various methods. For example, an easy method of collecting cells of the blood is by venipuncture. The biological sample may be collected from a performance animal, for example, a horse with suspected laminitis, a human athlete or camel with osteochondrosis, or a greyhound with subclinical cystitis.

Blood Sample

Ten ml of blood is drawn slowly (to prevent hemolysis) from the vein of an animal (jugular vein in a horse and camel, veins on the forearm/limb of humans and dogs) into a 1:16 volume of 4% sodium citrate to prevent clotting and the sample is mixed and then placed on ice. The sample is centrifuged at 3000 RPM at 4° C. for 15 minutes and white blood cells (WBC) (commonly called the “buffy coat”) are removed from the interface between plasma and red blood cells (RBC) into a separate tube using a pipette. The WBCs are then treated with at least 20 volumes of 0.8% ammonium chloride solution to lyse any contaminating RBC and re-centrifuged at 3000 RPM at 4° C. for 5 minutes. The pelletted WBCs are then washed in 0.9% sodium chloride, re-centrifuged, and kept on ice. The cell pellet is then used directly in RNA extraction.

Non-Blood Biological Fluid Sample

A fluid sample, for example, sputum, urine, bronchial or nasal lavages, joint fluid, peritoneal fluid or thoracic fluid, is centrifuged at 3000 RPM at 40° C. for 20 minutes to collect cells. Samples comprising large amounts of mucous are treated with a mucolytic agent such as dithiothretol prior to centrifugation. A cell pellet is then washed in 0.9% sodium chloride, re-centrifuged and the cell pellet is used directly in RNA extraction.

Tissue Biopsy

A tissue biopsy is frozen in dry ice or liquid nitrogen and crushed to powder using a mortar and pestle. The frozen tissue is then used directly in RNA extraction.

### STEP 2

RNA Isolation and Preparation

RNA Isolation

Total RNA and/or mRNA is isolated from a biological sample. Use of isolated mRNA rather than total RNA may provide results with less background and improved signal.

RNA is commonly isolated by skilled persons in the art, and examples of some methods for isolating RNA are described below.

Commercially available kits, for example, Qiagen RNA and Direct RNA extraction kits, and RNA extraction kits produced by Invitrogen (formerly Life Technologies) and Amersham Pharmacia Biotech herein incorporated by reference, may be used by following the manufacturer's instructions. Key elements of these extraction protocols include use of an appropriate amount of sample, protection of the sample from RNAse contamination, elution of the sample from a column at 70° C. and quantitation and quality checking in a separide (Invitrogen) 0.7% gel and using OD 260/280. About 0.2 gm (wet weight) of pelleted white blood cells or tissue is required for each mRNA extraction which will yield about 1-2 μg of mRNA. Disposable gloves should be worn throughout the procedure, with frequent changes. Both the column and solution used for elution should be at 70° C.

RNA quantification and assessment of RNA size and quality include standard gel electrophoresis methods of running a small quantity of an RNA sample on an agarose gel with known standards, staining the gel with for example ethidium bromide to detect the sample and standards and comparing relative intensities and size of standard RNA and sample RNAs. Alternatively, or in addition, RNA concentration in a solution may be determined by measuring absorbance at 260/280 nm in a spectrophotometer relative to known standards and calculated using known formulas.

cDNA Synthesis and Labelling

RNA prepared as described above may be synthesised to cDNA and labelled resulting in a labelled probe using kits provided by suppliers such as Amersham Pharmacia Biotech, Invitrogen, Stratagene or NEN, herein incorporated by reference. For example, a typical reaction may comprise: template RNA, an oligo-dT primer and/or gene-specific primers, reverse transcriptase enzyme, deoxyribonucleic triphosphates (dNTP), a suitable buffer, and a label incorporated into at least one of the dNTPs. Such a reaction when combined with a method of amplifying the resultant cDNA is referred to as RT-PCR (reverse transcriptase-polymerase chain reaction). A specific example is provided below, but it should be noted that other methods of incorporation of label into DNA can be used and that such methods are under constant review and improvement, for example some methods include the incorporation of amino-allyl dUTP and subsequent coupling of N-hydroxysuccinate activated dye to increase the specific labelling of the DNA.

To anneal primer(s) to template RNA, mix 2 μg of mRNA or 50-100 μg total RNA from respective test sample (Cy3) and reference sample (Cy5) in separate tubes with 4 μg of a regular or anchored oligo-dT primer or gene-specific primers in a total volume of 15 μl (using purified water to make up the volume). (Regular oligo dT is 5′-TTT TTT TTT TTT TTT TTT TTT, anchored oligo dT is 5′-TTT TTT TTT TTT TTT TTT TTV N-3′), (where V=A, C or G; and N=A, C, G or T). Heat mixture to 65° C. for 10 min and cool on ice. Add 15.0 μl of reaction mixture to respective Cy3 and Cy5 reactions.

The reaction mixture comprises of the following: 6.0 ul of 5× first-strand buffer, 3.0 μl of 0.1M DTT, 0.6 ul of unlabeled dNTPs, 3.0 ul of Cy3 or Cy5 dUTP (1 mM, Amersham), 2.0 ul of Superscript II (Reverse transcriptase 200 U/μL, Life Technologies) made to 15 μl with pure water. Unlabelled dNTPs are sourced from a stock solution consisting of 25 mM dATP, 25 mM dCTP, 25 mM dGTP, 10 mM dTTP. 5× first-strand buffer consists of 250 mM Tris-HCL (pH 8.3), 375 mM KCl, 15 mM MgCl₂). The mixture is incubated at 42° C. for 1 hr. Add an additional 1 μl of reverse transcriptase to each sample. Incubate for an additional 0.5-1 hrs. Degrade the RNA and stop the reaction by adding 15 μl of 0.1N NaOH, 2 mM EDTA and incubate at 65-70° C. for 10 min. If starting with total RNA, degrade the RNA for 30 min instead of 10 min. Neutralize the reaction by adding 15 μl of 0.1N HCl. Add 380 μl of TE (10 mM Tris, 1 mM EDTA) to a Microcon YM-30 column (Millipore).

Next add 60 μl of Cy5 probe and 60 μl of Cy3 probe to the same microcon. Centrifuge the column for 7-8 min. at 14,000×g. Remove flow-through and add 450 μl TE and centrifuge for 7-8 min. at 14,000×g (washing step). Remove flow-through and add 450 μl 1×TE, 20 μg of species-specific Cot1 DNA (20 ug/ul, Life Technologies for human—Cot1 DNA is genomic DNA that has been denatured and re-annealed such that the concentration of the DNA and the time of re-annealing multiplied equals 1. Methods for making Cot1 DNA are common in the art), 20 μg polyA RNA (10 μg/ul, Sigma, #P9403) and 20 μg tRNA (10 μg/ul, Life Technologies, #15401-011). Centrifuge 7-10 min. at 14,000×g. The probe needs to be concentrated such that with the addition of other solutions required for hybridisation the volume is not excessive, or is suitable for use with a desired slide and cover slip size. Invert the microcon into a clean tube and centrifuge briefly at 14,000 RPM to recover the probe.

A nucleic acid may be labelled with one or more labelling moieties for detection of hybridised labelled nucleic acid (ie. probe) and target nucleic acid complexes. Labelling moieties may include compositions that can be detected by spectroscopic, photochemical, biochemical, immunochemical, optical or chemical means. Labelling moieties may include radioisotopes, such as ³²P, ³³P or ³⁵S, chemiluminescent compounds, labelled binding proteins, heavy metal atoms, spectroscopic markers, such as fluorescent markers and dyes, magnetic labels, linked enzymes, and the like. Preferred fluorescent markers include Cy3 and Cy5, for example available from Amersham Pharmacia Biotech (as decribed above).

### Step 3

Arrays

One feature of the invention is an array comprising nucleic acids representing expressed genes from cells found in blood of a performance animal, for example a horse, human, camel or dog. The nucleic acids may be of any length, for example a polynucleotide or oligonucleotide as defined herein.

Each nucleic acid occupies a known location on an array. A nucleic acid target sample probe is hybridised with the array of nucleic acids and an amount or relative abundance of target nucleic acid hybridised to each probe in the array is determined.

High-density arrays are useful for monitoring gene expression and presence of allelic markers which may be associated with disease. Fabrication and use of high density arrays in monitoring gene expression have been previously described, for example in WO 97/10365, WO 92/10588 and U.S. Pat. No. 5,677,195, all incorporated herein by reference. In some embodiments, high-density oligonucleotide arrays are synthesised using methods such as the Very Large Scale Immobilised Polymer Synthesis (VLSIPS) described in U.S. Pat. No. 5,445,934, incorporated herein by reference.

Arrays for human are commercially available from companies such as Incyte, Research Genetics, and Affymetrix. Lion Bioscience recently announced forthcoming release of a dog microarray. These arrays typically comprise between 2,000 and 10,000 genes and are species specific. None are available for the horse or camel. Some of these genes are in multiple copies on the array and have not been fully annotated or given a true gene identity. Additionally, it is not known whether DNA on the array, when hybridised to a test sample, specifically binds to a single gene. This latter instance results from splice variants of RNA transcripts in tissues such that one gene may encode multiple transcripts.

Human and dog arrays (when available) can be used in methods described herein. However, these arrays are currently non-specific and include genes that are not expressed in blood cells of animals, and/or do not contain genes important in controlling the function of blood cells, and/or contain regions of genes that are not specific to blood cells.

Clones containing specific genes are available and can be purchased for human (and mouse) for use on arrays (for example from the IMAGE consortium). However, it is not possible to obtain specific clones for use on a blood-specific array without prior knowledge of what genes are expressed in blood cells. The IMAGE consortium also does not guarantee that the gene of interest is contained in the clone purchased.

Array Construction

Because of difficulties, problems and a likelihood of wasting financial resources to obtain a blood-specific DNA array, a method is provided herein which provides rapid and cost effective generation of species and tissue-specific DNA arrays for assessing nucleic acid expression in a sample. FIG. 3 shows steps for constructing an array in one embodiment.

Target Nucleic Acid Preparation

Biological samples are collected as described above. Samples comprising cells expressing as many genes of interest in relation to condition(s) of a performance animal are collected. For example, a sample comprising a mixture of nucleated blood cells from performance animals with conditions such as, osteochondrosis, laminitis, tendon soreness, bursitis, abcesses, inflammation, allergy, viral infection, parasite infection, asthma, etc.

Approximately 5 μg of mRNA is isolated from the biological sample (typically 1 gm wet weight) using mRNA isolation kits or the protocol described above. Concurrently, 5 μg of mRNA is isolated from umbilical cord blood, and/or early stage foetus. Cells and tissues contained within these sources would express genes that may not be expressed in the cells extracted from blood in the above example. Isolation of cytoplasmic mRNA from cells is preferred. This step involves rupturing the cells with a solution comprising detergent and/or chaotropic agent and salt such that cell nuclei and the nuclear membrane remain intact. The cell nuclei are pelleted by centrifugation and the supernatant is used for mRNA extraction. Protocols for this procedure are available as part of mRNA isolation kits (eg available by Qiagen). These mRNAs may be used to construct cDNA libraries. Kits for the construction of cDNA libraries are available from companies including Stratagene and Invitrogen (eg Uni-ZAP XR cDNA synthesis library construction kit #200450). The library preferably should be constructed such that the orientation of the cDNA in the vector is known, that the mRNA is primed using oligo dT, the vector is capable of receiving a nucleic acid insert up to 10 kb and that purification of DNA suitable for DNA sequencing is possible and easy. By following the manufacturer's instructions and paying particular attention to the quality of mRNA used and the size fractionation of cDNA (greater than 0.7 kb), a quality library containing enough viruses (>1×10⁶) with insert sizes >0.7 kb can be generated.

Plasmids generated from such a library can be DNA sequenced using protocols that are well established in the art and are available, for example, from Applied Biosystems. Briefly, a mix of 0.5 μg of plasmid DNA, 3.2 pmol of a primer that hybridises to the vector DNA (eg M13-21, or M13 reverse primer), thermostable DNA polymerase, dNTP and labelled dNTP is subjected to a routine PCR procedure to generate fragments of DNA that can be separated by gel electrophoresis and using machinery such as that available from Applied Biosystems (eg a 3700 DNA sequencer). Generated DNA sequence data (chromatogram) is assessed and manually called using a computer program such as Chromas™ The raw DNA sequence data can then be loaded into a database where comments (annotation) on the sequence can be made, such as quality, length of poly A sequence (should there be one), BLAST search results, highest homology in Genbank, clone identity, other entries in Genbank.

Subjective factors influencing whether a nucleic acid should be used on an array include quality and confidence of the DNA sequence, a Genbank homology score with identified nucleic acids, evidence of a poly-A tail (indicative of a translated transcript), uniqueness of the 3′ sequence data (compared to both Genbank and an in-house database of clone sequences).

Nucleic acid primers can be selected using a program such as Primer 3 available via the Internet (www-genome.wi.mit.edu/cgi-bin/primer/primer3). The selected primers may be used for amplifying a nucleic acid, for example by PCR, or directly applied to an array. Uniqueness of a nucleic acid can be tested by performing additional BLAST searches on Genbank and an in-house database. Primers are preferably designed such that melting temperatures are similar, and amplification products are of a similar nucleic acid length. Primers for PCR are generally between 18 and 25 nucleotide bases long. Primers for direct use on a microarray are preferably between 50 and 80 nucleotide bases long. Both the amplification product and the single primer should hybridise to DNA that uniquely identifies a gene transcript. Specific programs using various formulas are available for calculating the melting temperature of various lengths of DNA (eg Primer 3).

Nucleotide sequences may be compared with an existing database, for example Genbank, to determine a previously provided name, tissue expression, timing of expression, biochemical pathway, cluster membership, and possible function or cellular role of an expressed nucleic acid. In addition, a nucleic acid fragment may be used as a probe to isolate a full-length nucleic acid which may encode a gene which is associated with a particular disease or condition. Further, identified nucleic acids may be used to isolate homologues thereof, inclusive of orthologues from other species. An identified nucleic acid may also be cloned into a suitable expression vector to produce an expressed polypeptide in vitro, which may be used, for example as an antigen in generating antibodies. The antibodies may be used for developing specific diagnostic assays or therapies, for three-dimensional protein structure such as X-ray crystallographic studies, or for therapeutic development.

An array may comprise any number of different nucleic acids, but typically comprises greater than about 100, preferably greater than about 1,000, more preferably greater than about 5,000 different nucleic acids. An array may comprise more than 1,000,000 different nucleic acids. Each nucleic acid is preferably represented more than once for scanning internal comparison and control. Preferably, the nucleic acids are provided in small quantities and are gene-specific and/or species-specific usually between 50 and 600 nucleotides long, arranged on a solid support. The nucleic acids may be dotted onto the solid support. A typical array may have a surface area of less than 1 Cm², for example a microarray.

A nucleic acid can be attached to a solid support via chemical bonding. Furthermore, the nucleic acid does not have to be directly bound to the solid support, but rather can be bound to the solid support through a linker group. The linker groups may be of sufficient length to provide exposure to the attached nucleic acid. Linker groups may include ethylene glycol oligomers, diamines, diacids and the like. Reactive groups on the solid support surface may react with one of the terminal portions of the linker to bind the linker to the solid support. Another terminal portion of the linker is then functionalised for binding the nucleic acid. A solid support may be any suitable rigid or semi-rigid support, including charged nylon or nitrocellulose, chemically treated glass slides available from companies such as NEN, Corning, S&S, membranes, filters, chips, slides, wafers, fibers, magnetic or nonmagnetic beads, gels, tubing, plates, polymers, microparticles and capillaries. The solid support can have a variety of surface forms, such as wells, trenches, pins, channels and pores, to which the nucleic acids are bound. Preferably, the solid support is optically transparent.

The array may be constructed using an “arraying machine” manufactured by companies for example Molecular Dynamics, Genetic Microsystems, Hitachi, Biorobotics, Amersham, Corning. Source materials for this machine include microtitre plates comprising nucleic acids representative of unique genes. An array element may comprise, for example, plasmid DNA comprising nucleic acids specific for a gene sequence, an amplified product using gene-specific or non-specific primers and template DNA or RNA, or a synthesised specific oligonucleotide or polynucleotide. Array elements may be purified, for example, using Sephacryl-400 (Amersham Pharmacia Biotech, Piscataway, N.J.), Qiagen PCR cleanup columns, or high performance liquid chromatography (for oligonucleotides).

Purified array elements may be applied to a coated glass substrate using a procedure described in U.S. Pat. No. 5,807,522, incorporated herein by reference. By other example, DNA for use on Corning amino-silane coated slides (CMT-GAPS™) is re-suspended in 3×SSC to a concentration of 0.15-0.5 μg/μl and then used directly in an arraying machine in 96 or 384-well plates.

An example for preparing an array element is provided by the manganese superoxide dismutase gene. A clone comprising a nucleic acid insert is prepared and isolated as described above. The clone is sequenced to identify the nucleotide sequence. A BLAST search using the identified nucleotide sequence is performed to determine homology of the cloned nucleic acid with nucleic acids in a database, for example GenBank. Identification of nucleotide sequence homology with superoxide dismutase genes stored in the database provides a level of confidence that the clone comprises at least in part a gene for superoxide dismutase for the horse. A gene sequence unique to superoxide dismutase for the horse can then be determined by performing further BLAST searches. Unique primers can be designed to amplify a nucleic acid using PCR and the clone DNA, or genomic DNA from the same species as a template. Purified amplification product can be directly attached to an array and thereby act as a target for a complementary labelled nucleic acid probe in the test and reference samples. Alternatively, a unique sequence can be determined and an oliognucleotide manufactured and purified for direct use on an array.

The array may comprise negative and positive control samples (preferably as duplicates or triplicates) such as nucleic acids from species different from a sample being tested (negative controls) and various nucleic acids (representative of RNAs) that are found in all tissues as a constant and known quantity (positive controls). These controls are identified and used by the array reader to provide data on true signal (ie. Specific hybridisation between probe and target) and noise (ie. Non-specific hybridisation between probe and target) and average intensity from multiple reads of several different locations for each nucleic acid attached to the array.

A test sample and a reference sample are simultaneously assayed on the array. The reference sample may comprise mRNA from multiple sources, such that most, preferably all of the nucleic acids on the array are represented in the test sample, and can be used by the array reader as a non-zero standard and for comparison with an average of the read-outs from the test sample. A relative intensity for each gene on the array can be calculated.

The relative abundance of expression of each gene in a sample can also be calculated using controls within the array, such as certain genes expressed in a tissue at a constant level under all conditions.

The interpreted array may highlight only a few genes that are substantially different in expression between a test and reference sample. Alternatively, the overall pattern of expression may provide a “fingerprint” to characterise the way in which the original cells have responded to a particular condition of a performance animal. For example, the gene for superoxide dismutase may be the only gene up-regulated in a particular condition, especially in conditions of inflammation, or a large number of genes may be up- and down-regulated in various conditions.

The arrangement of nucleic acids on the array may be periodically changed and these arrays are then assigned a particular batch code which corresponds to a specific array comprising a specific nucleic acid arrangement. The ability to change the arrangement of nucleic acids on the array and knowledge of the exact arrangement may prevent other people from generating a database using the arrays produced by the present invention. Using a batch code also enables tracking of manufacturers of the arrays in regards to the number of arrays produced. The batch code further enables validation of a user of the communication network or “internet” diagnostic method and system. An array manufacturer providing an array for use with the method of the invention will only be provided with a limited quantity of nucleic acid for each gene to produce the arrays and will not be informed of the DNA sequences or gene identity. Primers and/or primer sequence in relation to genes or plasmid DNA need not be provided and preferably is not provided to a manufacturer of arrays. In this way, plasmids, primer sequences, gene arrangement on the array and numbers of arrays produced may be keep as a trade secret. Accordingly, a competitor cannot use genomic DNA to produce their own arrays (using primers determined by the present invention), or use an array prepared in accordance to the invention on performance animals to generate diagnostic databases.

An example of how an array may be prepared and analysed is described in Eisen and Brown (Methods in Enzymology, 1999, 303 179) and in U.S. Pat. No. 6,114,114, herein incorporated by reference. Chapter 22 of Ausubel et al. supra also describes methods and apparatus for use with arrays and is herein incorporated by reference.

Control samples may be respectively labelled in parallel with a test and reference sample. Quantitation controls within a sample may be used to assure that amplification and labelling procedures do not change a true distribution of nucleic acid probes in a sample. For this purpose, a sample may include or be “spiked” with a known amount of a control nucleic acid which specifically hybridises with a control target nucleic acid. After hybridisation and processing, a hybridisation signal obtained should reflect accurately amounts of control nucleic acid added to the sample. For such purposes, a microarray may have internal controls, for example a nucleic acid encoding a common gene expressed by the performance animal with known expression levels and a nucleic acid encoding a gene from another species that is known not to hybridise to the test or reference sample. To improve sensitivity and specificity of the assay, blocking agents such as Cot DNA from the tested species may also be used.

### Step 4

Hybridising Sample Nucleic Acid Probes with an Array

Nucleic acid probes may be prepared as described above from a biological sample from a performance animal that has been assessed concurrently by physical inspection and/or blood tests or other method. Nucleic acid probes from preferably about 1,000 normal animals are previously hybridised to arrays, and a reference range for each of the genes on the array is calculated and used as a normal reference range (for example a 95% population median). Results from a test sample from a test animal can be compared with the same genes as the normal reference to determine if the test sample falls within the normal reference range. Further, nucleic acid probes may also be prepared from biological samples from animals with overt disease, various progressive stages of disease, hitherto undiagnosed or unclassified conditions or stages of such conditions, animals treated with known amounts of drugs (legal or otherwise), animals suspected of being treated with drugs (legal or otherwise), animals under specific exercise regimes for the sake of performance, animals subjected to (intentional or not) various nutritional states and/or environmental conditions. Databases of information from the use of such samples and arrays are created such that test samples can be compared. The database will then contain specific patterns of gene expression for particular conditions.

Prior to hybridisation, a nucleic acid probe may be fragmented. Fragmentation may improve hybridisation by minimising secondary structure and/or cross-hybridisation with another nucleic acid probe in a sample or a nucleic acid comprising non-complementary sequence. Fragmentation can be performed by mechanical or chemical means common in the art.

A labelled nucleic acid probe may hybridise with a complementary nucleic acid located on an array. Incubation conditions may be adjusted, for example incubation time, temperature and ionic strength of buffer, so that hybridisation occurs with precise complementary matches (high stringency conditions) or with various degrees of less complementarity (low or medium stringency conditions). High stringency conditions may be used to reduce background or non-specific binding. Specific hybridisation solutions and hybridisation apparatus are available commercially by, for example, Stratagene, Clontech, Geneworks.

A typical method entails the following:

Adjust probe volume (prepared as above) to a value indicated in the “Probe & TE” column below according to the size of the cover slip to be used and then add the appropriate volume of 20×SSC and 10% SDS.

| Cover Slip | Total Hyb | Probe & TE | | |
|---|---|---|---|---|
| Size (mm) | Volume (μl) | (μl) | 20x SSC (μl) | 10% SDS (μl) |
| 22 × 22 | 15 | 12 | 2.55 | 0.45 |
| 22 × 40 | 25 | 20 | 4.25 | 0.75 |
| 22 × 60 | 35 | 28 | 5.95 | 1.05 |

20xSSC is 3.0 M NaCl, 300 mM NaCitrate (pH 7.0).

Denature the probe by heating it for 2 min at 100° C., and centrifuge at 14,000 RPM for 15-20 min. Place the entire probe volume on the array under the appropriately sized glass cover slip. Hybridize at 65° C. (temperatures may vary when using different hybridisation solutions) for 14 to 18 hours in a custom slide chamber (for example a Corning CMT hybridisation chamber #2551).

Washing the Array

After hybridisation, the array is washed to remove non-specific probe and dye hybridisation. Wash solutions generally comprise salt and detergent in water and are commercially available. The wash solutions are applied to the array at a predetermined temperature and can be performed in a commercially available apparatus. Stringency conditions of the wash solution may vary, for example from low to high stringency as herein described. Washing at higher stringency may reduce background or non-specific hybridisation. It is understood that standardisation of this step is required to produce maximum signal to noise ratio by varying the concentration of salt used, whether detergent is present (SDS), the temperature of the wash solution and the time spent in the wash solution.

A typical wash protocol consists of removing the slide from a slide chamber, removing the cover slip and placing the slide into 0.1% SSC (recipe provided above) and 0.1% SDS at room temperature for 5 minutes. Transfer the slide to 0.1% SSC for 5 minutes and repeat. Dry the slide using centrifugation or a stream of air. Equipment is available to enable the handling of more than one slide at a time (for example, slide racks).

### STEP 5

Reading the Array

After removal of non-hybridised probe, a scanner or “array reader” is used to determine the levels and patterns of fluorescence from hybridised probes. The scanned images are examined to determine degree of hybridisation and the relative abundance of each nucleic acid on the array. A test sample signal corresponds with relative abundance of an RNA transcript, or gene expression, in a biological sample.

Array readers are available commercially from companies such as Axon and Molecular Dynamics. These machines typically use lasers at different frequencies to scan the array and to differentiate, for example, between a test sample (labelled with one dye) and the control or reference sample (labelled with a different dye). For example, an array reader may generate spectral lines at 532 nm for excitation of Cy3, and 635 nm for excitation of Cy5.

A relative quantity of RNA may be calculated by the array reader and computer for respective nucleic acids on the array for respective samples based on an amount of dye detected, average of duplicate samples for respective genes and subtraction of background noise using controls. The reader is pre-programmed to perform such calculations and with information on the location of each nucleic acid on the array such that each nucleic acid is given a readout value. Controls or reference samples providing a readout for particular nucleic acids that falls within standard ranges ensures correct integrity of the array and hybridisation procedures. Programs typically generate digital data and format it for transmission.

### Step 6

Automated Transfer of Digital Data to a Central Database

Generated data is transmitted via a communications network to a remote central database. A user having access to the microarray readout enters information in relation to a test sample into a standard diagnostic form such that it can be digitalised. The information will include clinical appraisal and blood profile results. The format of such information is standard globally such that details on clinical conditions are based on numerical input and each field of entry can be digitalised. For example, body temperature field could be number 0001, a recorded temperature within normal range would receive the number 0, 0.5° C. above what is considered to be the normal range for that species would receive a number 5, 1° C. above normal range would receive 10. Some examples of conditions that may be scored or rated in such a fashion are provided below.
a) Body temperature.
b) Integument: eyes, sores, abcesses, wounds, insects/parasites, allergy, infection.
C) Cardio/Respiratory: eyes, nasal discharge, rales, viral/bacterial infection, allergy, chronic obstructive pulmonary disease, cough/wheeze, crepitous sounds in the thorax, epistaxis, auscultation sounds, heart sounds, capillary refill, mucous membrane colour.
d) Gastrointestinal: diarrhoea, colic/stasis, parasites, appetite level, drenching time and dose.
e) Reproductive: stage of pregnancy, abortion, inflammation, discharges.
f) Musculoskeletal: lameness, laminitis, bone or shin soreness, muscle soreness or tying up, tendon or ligament affected, level of pain, X-ray data, scintigraphy data, CAT scan data, bursitis, bruising, cramping or “tying up”.
g) Blood test results: biochemistry, immunology, serology (viral, bacteriological, hormone levels), cell counts, cell morphology, pathologist interpretation.
h) Other diagnostic test results: X-ray, biopsy, histopathology, CAT scan, MRI, bacteriology, virology.
i) Other data: Season (date), location, male or female, vaccination history, body score (fitness and fat), fitness level.

The user also ensures that array results (that may for example be automatically collected from a reader), array specifications, data mining specifications, level of interpretation required and the clinical information are entered and correspond to the same animal and the same sample. The form is transmitted electronically to a central database and recognised as an individual accession or request by the database. The central database recognises the user (using for example digital certificates), the user recognises the central database, the array batch code and gene array order are verified, and the user is allowed access (which may be automatic) and automatic processing of the request is performed if security and billing information are adequate. The processing involves specific mining of central data and specific user requested information is retrieved and resent automatically.

The above steps may be automated so that a user need not be present to perform the tasks. In an automated embodiment of the invention, data from a microarray reader may be transmitted via a communications network directly to a server which is connected to a central database. Additional information could be input by the user at a processor which is also linked to the microarray reader.

Automated Data Mining Using Sent Data

A central database interprets the array specifications (eg. nucleic acid order on a microarray), decodes the information transmitted, determines nucleic acid expression level in a biological sample and compares the expression level and patterns of expression with known standards or reference range. Various levels of database interpretation may be applied to the data transmitted, depending on the user requirements. Clusters of genes may be up-regulated or down-regulated in certain conditions and the database makes automated correlations to specific conditions by accessing various levels of database information.

Levels of database may include:

Unique gene sequences (eg 3′ and 5′ EST sequence of genes)
Gene identity, homologous genes, tissue expression, keywords, function, cellular role, gene clusters
Primer sequences used to generate amplification products (eg two primer sequences used to uniquely amplify the gene for gamma interferon in a particular species)
Microarray construction and format (eg coded information on array manufacture batch and identification of genes and position on the array)
Blood profile and clinical data associated with particular conditions (eg standard clinical information and IDEXX-machine generated blood profile data)
Array data for normal status (eg 95% median range for normal animals)
Array data for various overt diseases (eg joint inflammation)
Array data for stages of various overt diseases (eg pre-clinical, clinical and recovery stages)
Array data for the influence of various classes of drugs, legal or otherwise, of known administration and dose, or unknown administration or dose (eg various steroids)
Array data for the response to known and various levels of drugs used as a therapy (eg various anti-inflammatory medication at specific doses for a specific condition)
Array data for the response to exercise and various training regimes
Array data for the response to nutrition and various feeding regimes
Array data for the response to the environment so as to possibly determine influence of during various seasons, or allergens or feed types.

Each successive level relies on at least one previous level of database to allow for interpretation. The database may be built over time and more intensive searching of the database may incur a greater cost. As the database grows, changes may be made to the above methodology to increase the sensitivity of the detection of variation in expression of condition-specific genes—this could include the use of condition-specific arrays or condition-specific primers. This process is iterative, such that specific genes are correlated to specific conditions, and the detection of variations in these genes becomes more sensitive and specific through the use of various modifying processes through the procedure (eg. the use of gene-specific primers for the amplification and labelling of cDNA from RNA, and the selection of limited numbers of genes on a disease- or condition-specific array).

### Step 7

Standardised Electronic Reporting

The database reports back electronically to a remote user, either automatically or with a level of human intervention.

Information sent might include:

Individual genes up-regulated or down-regulated (for example, with laminitis or joint capsule inflammation or bursitis, a report on the up-regulation of genes such as interleukin-3, manganese superoxide dismutase, Groα, metalloproteinase matix-metallo-elastase, ferritin light chain may have some correlation to tissue inflammation, and down-regulation of genes such as insulin-like growth factor and its receptor may be correlated to recovery from such a condition). The identity of these genes cannot be predicted to be associated to any condition unless the above described methodology is used and databases on relative expression of genes for particular conditions have been compiled.

The overall pattern of gene expression and any correlation to particular conditions. For example, animals in heavy training may have a gene “fingerprint” that is different to animals being spelled or taking a spell from training.
Individual pattern of gene expression (ie. the shape of the gene expression pattern over a time course or multiple samples taken over a period may change as an animal recovers from a condition)
Changes to a pattern of gene expression, gene expression profile or level for a single animal over a time period or for successive tests.
Clusters of genes up-regulated or down-regulated in a particular condition
Pathways of genes up-regulated or down-regulated in a particular condition
Correlations between genes up-regulated or down-regulated and known conditions, or stage of condition, or influence
Known therapies to ameliorate the condition or enhance desired effects
Pathologist written interpretation

Throughout the specification the aim has been to describe the preferred embodiments of the invention without limiting the invention to any one embodiment or specific collection of features. It would therefore be appreciated by those of skill in the art that, in light of the instant disclosure, various modifications and changes can be made in the particular embodiments exemplified without departing from the scope of the present invention. For example, the examples described herein may be used with performance animals other than horse, for example human, dog and camel. The methods may also be used with non-performance animals, including for example plants and insects.

All references, inclusive of patents, patent applications, scientific documents and computer programs, referred to in this specification are herein incorporated by reference in its entirety.

1. A method for assessing a condition of a performance animal including the steps of:
(a) determining in a sample from a performance animal a relative abundance of a target nucleic acid normalised to a reference nucleic acid and providing the relative abundance of the target nucleic acid as a digital signal;
(b) accessing a remotely located database comprising digital information in relation to relative abundance of the target nucleic acid which corresponds to a particular condition of the performance animal;
(c) correlating the digital signal of step (a) with the digital information of step (b) thereby identifying a particular condition of the performance animal; and
(d) reporting the particular condition of the performance animal.

2. The method of claim 1 whereby the step of determining the relative abundance of the target nucleic acid includes the steps of:
(i) detecting a hybridised complex formed by at least one target nucleic acid and a complementary nucleic acid located on a solid support to provide a digital target sample signal;
(ii) detecting a hybridised complex formed by at least one reference nucleic acid and a complementary nucleic acid located on a solid support to provide a digital reference sample signal; and
(iii) comparing the digital target sample signal of step (i) and the digital reference sample signal of step (ii) to provide a digital signal of relative abundance of the target sample.

3. The method of claim 2 whereby the complementary nucleic acids of step (i) and step (ii) comprise a same or homologous nucleotide sequence.

4. The method of claim 2 whereby the hybridised complex in step (i) is detected by labelling the target nucleic acid.

5. The method of claim 4 whereby the labelled nucleic acid is labelled with Cy3 or Cy5.

6. The method of claim 4 whereby the labelled nucleic acid is cDNA.

7. The method of claim 2 whereby the hybridised complex in step (ii) is detected by labelling the reference nucleic acid.

8. The method of claim 7 whereby the labelled nucleic acid is labelled with Cy3 or Cy5.

9. The method of claim 7 whereby the labelled nucleic acid is cDNA.

10. The method of claim 2 whereby the respective target nucleic acid and reference nucleic acid are concurrently hybridised with respective complementary nucleic acids.

11. The method of claim 2 whereby the target nucleic acid and the reference nucleic acid have a same or homologous nucleotide sequence and are respectively labelled with different labels.

12. The method of claim 2 whereby the solid support is an array.

13. The method of claim 12 whereby the array is a microarray.

14. The method of claim 1 wherein the database is accessible via a communications network.

15. The method of claim 14 wherein the communications network comprises the Internet, an intranet, an extranet or wireless means.

16. The method of claim 1 wherein the performance animal is a mammal.

17. The method of claim 16 wherein the mammal is human, horse, dog or camel.

18. The method of claim 1 wherein the condition enhances, hinders, impedes or does not change an expected ability of the performance animal.

19. The method of claim 18 wherein the condition comprises normal, pre-clinical disease, overt disease, progress and/or stage of disease, undiagnosed or unclassified conditions, presence of drugs, response to drugs, response to exercise, response to vaccines, therapies, nutritional states and response to environmental conditions.

20. The method of claim 19 wherein the disease comprises laminitis, lameness, viral disease, colic, gastritis, gastric ulcers, respiratory ailments and epistaxis.

21. A diagnostic system comprising:
(A) a microarray comprising respective nucleic acids complimentary to a target nucleic acid and reference nucleic acid;
(B) a microarray reader that detects hybridised complexes formed respectively by the target nucleic acid and the reference nucleic acid with their complimentary nucleic acids and generates a digital signal;
(C) a database storing information in relation to relative abundance of the target nucleic acid corresponding to a particular condition of a performance animal;
(D) a diagnostic server that receives the digital signal and correlates the digital signal with information in the database to identify said particular condition and reports said particular condition; and
(E) a means for communicating between the microarray reader and the diagnostic server.

22. The diagnostic system of claim 21 wherein the microarray reader determines relative abundance of the target nucleic acid normalised to the reference nucleic acid and generates a digital signal for the relative abundance of the target nucleic acid.

23. The diagnostic system of claim 21 wherein the means of communication is a network.

24. The diagnostic system of claim 21 further comprising a display means to display the report.
